(19) United States
(12) Patent Application Publication
(10) Pub. No.: US 2025/0375183 A1
(43) Pub. Date: Dec. 11, 2025

(54) PATIENT FLUID MANAGEMENT SYSTEMS AND METHODS EMPLOYING INTEGRATED FLUID STATUS SENSING

(71) Applicant: Foundry Innovation & Research 1, Ltd., Dublin (IE)

(72) Inventors: Fiachra M. Sweeney, Dublin (IE); Hanson S. Gifford, III, Woodside, CA (US); Annette Kent, Dublin (IE); Conor M. Hanley, Dublin (IE)

(73) Assignee: Foundry Innovation & Research 1, Ltd., Dublin (IE)

(21) Appl. No.: 19/301,609

(22) Filed: Aug. 15, 2025

Related U.S. Application Data
(63) Continuation of application No. 17/245,020, filed on Apr. 30, 2021.
Application No. 17/245,020 is a continuation-in-part of application No. 17/162,857, filed on Jan. 29, 2021.
Application No. 17/162,857 is a continuation-in-part of application No. 16/271,798, filed on Feb. 9, 2019, now Pat. No. 11,564,596.
Application No. 17/162,857 is a continuation of application No. 15/549,042, filed on Aug. 4, 2017, now Pat. No. 10,905,393.
Application No. 16/271,798 is a continuation of application No. PCT/US2017/046204, filed on Aug. 10, 2017.
(60) Provisional application No. 62/115,435, filed on Feb. 12, 2015.
(60) Provisional application No. 62/157,331, filed on May 5, 2015.
(60) Provisional application No. 62/172,516, filed on Jun. 8, 2015.
(60) Provisional application No. 62/373,436, filed on Aug. 11, 2016.
(60) Provisional application No. 62/427,631, filed on Nov. 29, 2016.
(60) Provisional application No. 62/534,329, filed on Jul. 19, 2017.
(60) Provisional application No. 63/017,958, filed on Apr. 30, 2020.

(51) Int. Cl. A61B 8/08; A61B 5/00; A61B 5/107; A61B 8/00; A61B 8/12; A61B 90/00

(52) U.S. Cl. CPC A61B 8/0891; A61B 5/1076; A61B 5/6882; A61B 8/12; A61B 8/565; A61B 90/39; A61B 5/4836; A61B 5/4839; A61B 2090/3929; A61B 2090/3987; A61B 2090/3991

(57) ABSTRACT

Patient fluid management systems, particularly for use in treating patients at various stages of heart failure, are disclosed. Disclosed systems employ vascular dimension monitoring sensors to provide accurate, early, real-time estimation of circulating blood volume as an input metric to the system control, allowing for more accurate modulation of treatment based on the patient's current fluid volume state.

### RELATED APPLICATIONS

The present application is a continuation application of U.S. patent application Ser. No. 17/245,020, filed on Apr. 30, 2021, and entitled “Patient Fluid Management Systems and Methods Employing Integrated Fluid Status Sensing,” which application claims priority to U.S. Provisional Application No. 63/017,958, filed on Apr. 30, 2020, and entitled “Patient Fluid Management Systems and Methods Employing Integrated Fluid Status Sensing.”

U.S. patent application Ser. No. 17/245,020, is also a continuation-in-part application of U.S. Continuation patent application Ser. No. 16/271,798, filed Feb. 9, 2019, and entitled “Systems and Methods for Patient Fluid Management” (now U.S. patent Ser. No. 11,564,596 granted Jan. 31, 2023), which application is a continuation of PCT/US2017/046204, filed Aug. 10, 2017, which application claims priority to U.S. Provisional Patent Application No. 62/534,329, filed Jul. 19, 2017, and entitled “Wireless Vascular Monitoring Implants, Systems and Methods”; U.S. Provisional Patent Application No. 62/427,631, filed Nov. 29, 2016, and entitled “Wireless Vascular Monitoring Implants, Systems, Methods, and Software”; and U.S. Provisional Patent Application No. 62/373,436, filed Aug. 11, 2016, and entitled “Methods and Systems For Patient Fluid Management”.

U.S. patent application Ser. No. 17/245,020, is also a continuation-in-part application of U.S. Continuation patent application Ser. No. 17/162,857, filed Jan. 29, 2021, and entitled “Patient Self-Monitoring of IVC Volume for Early Heart Failure Warning Signs”; which is a continuation of U.S. Nonprovisional patent application Ser. No. 15/549,042 filed Aug. 4, 2017, and entitled “Implantable Devices and Related Methods for Heart Failure Monitoring” (now U.S. patent Ser. No. 10/905,393 granted Feb. 2, 2021); which application was a U.S. National Phase Application of PCT/US2016/017902 filed Feb. 12, 2016; which application claimed priority to U.S. Provisional Patent Application No. 62/172,516 filed Jun. 8, 2015, and entitled “Methods and Apparatus for Monitoring Patient Physiological Status Based on Inferior Vena Cava Volume”; U.S. Provisional Application No. 62/157,331 filed May 5, 2015, and entitled “Heart Failure Monitoring System and Method”; and U.S. Provisional Patent Application No. 62/115,435 filed Feb. 12, 2015, and entitled “Implantable Devices and Related Methods for Heart Failure Monitoring”.

Each of these applications is incorporated herein by reference in its entirety.

### FIELD

The present disclosure relates generally to therapy systems and methods for modulating patient fluid volume, and more specifically to patient fluid management systems and methods employing integrated fluid status sensing.

### BACKGROUND

Many medical treatment systems employed to treat a variety of health-related problems incorporate or rely on information about patient fluid status as a parameter in modulating treatment. Examples of such treatment systems include, but are not necessarily limited to, dialysis systems, ultra-filtration systems, diuresis systems, left ventricular assist devices (LVAD), renal flow modulating systems, and active drainage systems. Operation of other devices, such as drug pumps and nerve stimulation devices may also be modulated more accurately with accurate patient fluid status information.

### SUMMARY

In one implementation, the present disclosure is directed to an integrated patient fluid management system. The system includes an interventional device configured to effect a change in patient fluid state by delivery of an interventional therapy; a vascular dimension monitoring sensor configured to be positioned within a vascular lumen and to monitor changes in dimension of the vascular lumen resulting from changes in patient fluid state; and a control system configured to (i) communicate with the interventional device and the vascular dimension monitoring sensor and (ii) modulate the delivered interventional therapy in response to vascular dimension measurements as determined by the vascular dimension monitoring sensor.

In another implementation, the present disclosure is directed to an integrated patient fluid management system with assisted diuresis. The system includes a wireless implantable vascular dimension sensor monitor configured to be positioned within a vascular lumen to monitor changes in dimension of the vascular lumen resulting from changes in patient fluid state; processor-controlled infusion pump configured to deliver an interventional therapy comprising delivering at least one of a diuretic or an infusate to the patient through an infusion line in accordance with infusion rate and limit instructions set by the processor control; and a fluid status monitoring and infusion control system comprising a user interface, at least one processor, and memory, configured to: wirelessly communicate with the wireless implantable vascular dimension sensor and receive a signal therefrom indicative of changes in the monitored vascular dimension; derive current patient fluid state information from the received signals; receive a desired fluid loss goal input through the user interface and store desired fluid loss goal information in the memory; compare the current patient fluid state information with the fluid loss goal information to determine the interventional therapy directed to achieve the desired fluid loss goal; and instruct the processor-controlled infusion pump to deliver the interventional therapy to the patient at an infusion rate and limit set by the control system based on the comparison of current patient fluid state information with the fluid loss goal information.

In yet another implementation, the present disclosure is directed to an integrated patient fluid management method. The method includes setting a desired fluid loss goal by inputting the desired fluid loss goal into a system controller comprising a processor and memory; monitoring changes in dimension of a vascular lumen resulting from changes in patient fluid state using a wireless vascular dimension monitoring sensor implanted within the vascular lumen; determining changes in patient fluid status by wirelessly receiving and interpreting a signal from the vascular dimension monitoring sensor; administering a diuretic to the patient; and automatically administering a fluid to the patient by an infusion pump under control of the system controller until the desired fluid loss goal is reached.

### BRIEF DESCRIPTION OF DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 is a schematic depiction of generic systems of the present disclosure.

FIG. 2 is a schematic depiction of an embodiment of an assisted diuresis system according to the present disclosure.

FIG. 3A is a schematic depiction of an embodiment of a direct sodium removal system according to the present disclosure.

FIG. 3B is schematic depiction of an alternative embodiment of the direct sodium removal system with the addition of a sodium sensor.

FIG. 4 is a flow diagram depicting an embodiment of a process for direct sodium removal according to the present disclosure.

FIG. 5 is a schematic depiction of an embodiment of an IVC occlusion system according to the present disclosure.

FIG. 6 is a schematic depiction of an IVC occlusion system according to an embodiment of the present disclosure as deployed in a patient IVC.

FIG. 7 is a schematic depiction describing the body fluid compartments in the body and their respective and relative volumes, and an embodiment of a fluid management system, capable of monitoring and managing the intravascular and interstitial fluid spaces individually.

FIG. 8 is a block diagram depicting high-level operation of a fluid management system employing a lymphatic system assist pump according to further embodiments of the present disclosure.

FIG. 9 is a schematic depiction of a patient fluid management system employing a wirelessly controlled lymphatic assist pump, showing an enlarged detail of patient anatomy at the branch of the inner jugular and subclavian veins.

FIGS. 10A and 10B are schematic depictions of a patient treatment system including diaphragm stimulation with integrated fluid monitoring.

FIGS. 11A and 11B are schematic depictions of patient treatment systems including IVC or renal vein assist with integrated fluid monitoring.

FIGS. 12A and 12B are schematic depictions of a patient treatment system including a patch pump for diuretic or other therapeutic agent with integrated fluid monitoring.

### DETAILED DESCRIPTION

The present Applicant has previously developed and disclosed a number of different implantable, wireless sensors for determining patient fluid status based on direct measurement of a vascular dimension, which indicates geometry, namely cross-sectional area and distension or collapse of the vessel. This measurement of vessels, particularly of the inferior vena cava (IVC), has been demonstrated to relate directly to a patient's circulating blood volume. Therefore such measurements with these sensors can be used to estimate a patient's circulating blood volume. In particular, these sensors can be used to determine whether circulating blood volume is too high or too low, and whether circulating blood volume is increasing or decreasing.

Such implantable sensors are capable of long-term placement suitable for monitoring patients with chronic conditions. Examples of such implantable, wireless sensors are disclosed, for example, in U.S. patent application Ser. No. 15/549,042, filed Aug. 4, 2017 (U.S. Pat. No. 10,905,393, granted Feb. 2, 2021), and entitled “Implantable Devices and Related Methods for Heart Failure Monitoring” and U.S. patent application Ser. No. 16/177,183, filed Oct. 31, 2018 (U.S. Pat. No. 10,806,352, granted Oct. 20, 2020) and entitled “Wireless Vascular Monitoring Implants,” each of which is incorporated herein in its entirety. In other clinical situations, such as shorter term acute condition monitoring and in-hospital treatments, vascular dimension sensors for direct fluid state determination and monitoring may be catheter-based. Examples of such catheter-based sensors are disclosed, for example, in U.S. patent application Ser. No. 15/750,100, filed Feb. 2, 2018 (U.S. Publication No. US20180220992, published Aug. 9, 2018) and entitled “Devices and Methods for Measurement of Vena Cava Dimensions, Pressure and Oxygen Saturation,” which is incorporated herein in its entirety.

The present Applicant has also developed a new dialysis and other treatment systems to provide improved control and new treatment modalities based on and incorporating sensors as disclosed in the above-mentioned incorporated disclosures. Embodiments of such new systems are disclosed, for example, in U.S. patent application Ser. No. 16/271,798, filed Feb. 9, 2019 (US20190167188, published Jun. 6, 2019) and entitled “Systems and Methods for Patient Fluid Management” and International Patent Application No. PCT/IB2019/060669, filed Dec. 11, 2019 (WO2020/121221, published Jun. 18, 2020) and entitled “Dialysis Catheters with Integrated Fluid Status Sensing and Related Systems and Methods,” each of which is incorporated by reference in its entirety herein.

The present disclosure provides additional embodiments and details of further systems based on and employing the new sensor devices as described above so as to offer new treatment modalities and possibilities for enhanced patient outcomes through optimized or new treatments. The disclosed sensors, hereinafter referred to as Fluid Status Monitoring Devices or Systems, provide an accurate, early, real-time estimation of circulating blood volume. This metric provides valuable information that is integrated into specific treatment systems as described herein in order to optimize performance and, in some cases, provide new treatment modalities or possibilities. As described in detail in the incorporated prior disclosures, the disclosed Fluid Status Monitoring Devices and Systems include a number of different and varied new embodiments incorporating sensing means, such as passive sensors excited by an external reader and hardware that (locally/in the cloud) could compute results and then input into other systems, or active sensors powered locally that could then communicate either directly with other systems or indirectly through an implanted monitor to communicate with other systems. Other systems that a disclosed Fluid Status Monitoring Device may communicate with could be either implanted within the body or external to the body.

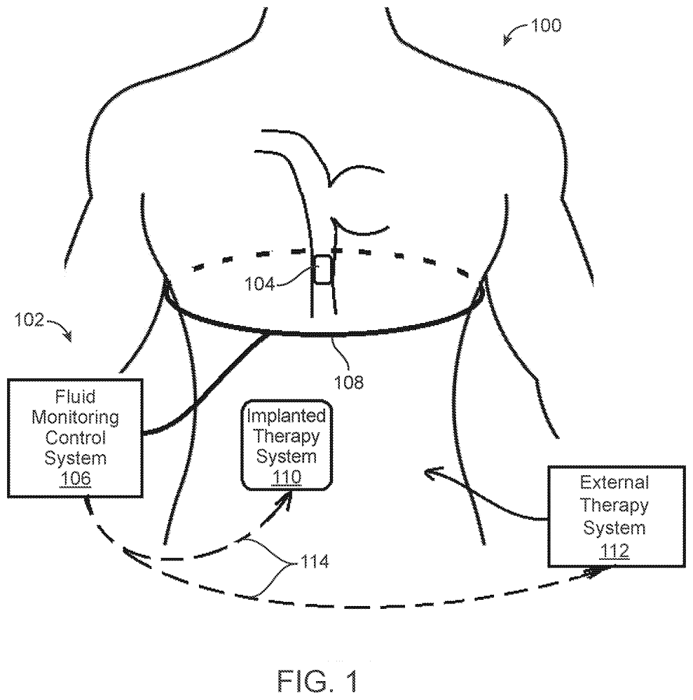

FIG. 1 schematically depicts a generic system according to the present disclosure. As shown therein a medical treatment system 100 of the present disclosure includes a Fluid Status Monitoring System 102 with a Fluid Status Monitoring Device 104 and Fluid Status Monitoring control system 106 as described above and in the incorporated applications. An external antenna 108 receives the vascular dimension signal from Fluid Status Monitoring Device 104, which is then processed into fluid status information by control system 106. Control system 106 communicates with one or more of implanted therapeutic devices or systems 110 and/or external therapeutic devices or systems 112 via communication channels 114, which may be wired or wireless. Therapies provided by systems 110 and 112 are modulated and/or optimized by their internal control systems based on patient fluid status information received from control system 106. Such information may be provided periodically or continuously to allow for real-time closed loop feedback control. An alternative embodiment could include a Fluid Status Monitoring Device 104 which communicates directly to the implanted therapeutic system 110 without the signal being first transmitted outside the body. Specific embodiments of various systems following this general arrangement are further described below.

### Assisted Diuresis

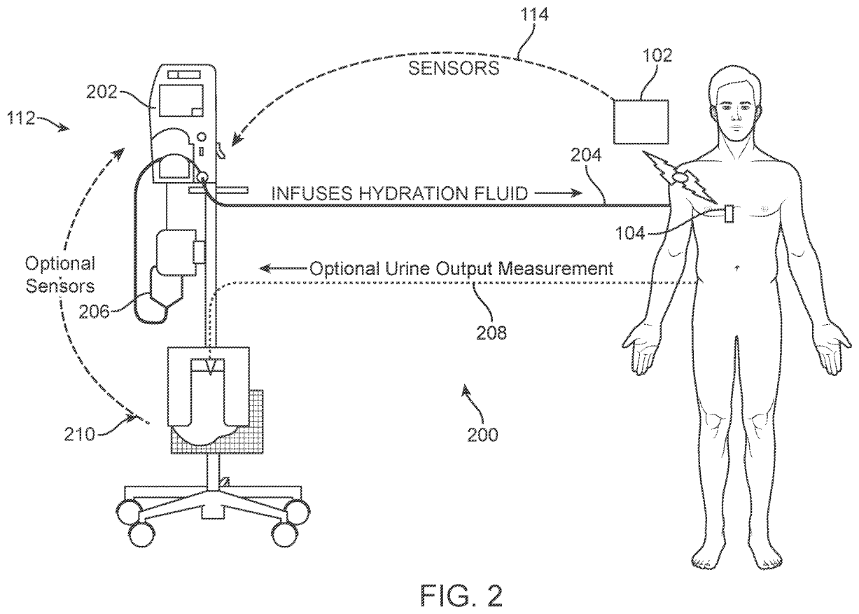

Systems have been proposed to assist with the physiology of diuresis by infusing fluid into the patient as diuresis occurs to maintain optimal renal function and ultimately increase the volume of fluid removed from the patient, as disclosed, for example, in U.S. Publication No. US2018/0326151, filed Jul. 11, 2018, and published on Nov. 15, 2018, entitled “Fluid Therapy Method.” Fluid Status Monitoring Devices and Systems as described herein-above may be integrated with these assisted diuresis systems such as disclosed in the foregoing published application. An assisted diuresis system according to the present disclosure, such as schematically depicted in FIG. 2, may be used in heart failure diuresis for the administration of saline while giving high doses of diuretics. Such treatments have been shown to increase the overall volume of urine extracted in heart failure patients and other patients in need of fluid balance treatments.

As illustrated in FIG. 2, in an improved diuresis system 200 according to the present disclosure, Fluid Status Monitoring System 102 as disclosed above provides accurate, early, real-time intravascular fluid status information via vascular dimension metric as determined by a Fluid Status Monitoring Device 104, thus allowing overall system 200 to determine the optimal level of circulating blood volume to maximize the urine output. In prior systems, urine output was measured by placing a Foley catheter, connecting it to a urine collection bag, and continuously measuring the weight of the urine collection bag. This measurement of urine output was then used as an indirect surrogate to manage the patient's circulating blood volume and overall fluid status. However, this is not an ideal surrogate because other factors are involved such that urine output may not always accurately reflect circulating blood volume of the patient. Moreover, the balance of fluid between the intravascular and extravascular spaces is unknown. This knowledge gap limits the ability of the system to operate optimally as neither intravascular nor extravascular fluid states are being measured. The lack of an accurate intravascular sensor, in particular, presents other challenges. For example, the initial fluid status of the patient is unknown and therefore there is a risk of hypovolemia or hypervolemia until the treatment protocol is well underway. Intravascular volume measurement might also help to manage the end of the treatment protocol, to assess when sufficient diuresis has occurred and prevent overtreatment. Also, in one typical implementation, a weight sensor is used to determine fluid output automatically as a control parameter, which can introduce further inaccuracies. Additional disadvantages include the fact that catheterization with a Foley catheter is a cumbersome, unpleasant additional requirement for the patient, that presents a risk of infection and additional care requirements for care providers.

In the improved system 200, shown in FIG. 2, circulating blood volume is accurately estimated in real-time, continuously if required, by Fluid Status Monitoring System 102. System 102 communicates with the external treatment system 112, which in this case may comprise processor-controlled infusion pump 202, which delivers the infusate via infusion line 204. Reservoir 206 may provide a known quantity of infusate to be delivered at the rate set by infusion pump 202 based on patient fluid status information as determined by Fluid Status Monitoring System 102 communicating through communication channels 114 with pump 202. The same or additional processor-controlled syringe pump(s) or infusion pump(s) also may deliver a diuretic such as Furosemide and/or other medicaments to increase or decrease the rate of diuresis. It should be recognized that the present embodiment does not preclude the use of a Foley catheter 208 as a further optional system element. In such cases, optional additional sensors 210 may be employed, such as a weight sensor, to determine urine output as a further parameter input to external system 112.

In general, as can be seen based on the example shown in FIG. 2, the disclosed Fluid Status Monitoring Systems and Devices may be employed as direct inputs to IV diuretic pumps, syringe pumps and other infusion devices to facilitate automated administration and control of IV diuretics. The ability of the disclosed Fluid Status Monitoring Systems to detect the onset of hypovolemia would allow the IV drug pump or other infusion device to be run at a higher rate in the initial phases of diuresis and then reduce the drug dosage and/or increase the infusion of saline or other fluids as hypovolemia is approached. This would maintain a euvolemic state while also maximizing the fluid removed and minimizing the total time of therapy and total dose of drugs administered to the patient.

### Acute Kidney Injury

Contrast induced nephropathy (CIN) occurs in some patients undergoing interventional or surgical procedures. Infusion technologies such as disclosed in U.S. Publication No. US2010274217, filed Jan. 14, 2010, and published on Oct. 28, 2010, and entitled “Fluid Replacement Device,” infuse saline and diuretics before and during procedures to increase the rate of urine output, thereby diluting the contrast in the kidneys and increasing the rate of contrast excretion. CIN is sometimes defined as a 25% increase in serum creatinine (SCr) from baseline. Possible treatments for CIN involve hydration treatments that infuse saline before and during procedures to dilute the contrast in the kidneys. Conventionally, as with assisted diuresis described above, a Foley catheter has been required to measure the urine output and match the infusion flow rate to maintain fluid balance. However, using a system such as described above and shown in FIG. 2, hydration treatment for CIN may be accomplished with more direct and more accurate assessment of the blood volume status of the patient. This would eliminate the need for a Foley catheter and provide the opportunity to optimize the infusion rate.

### Direct Sodium Removal

In some patients with fluid volume overload due to heart failure, there may be excess sodium as well as excess water. Removing both at once can be challenging. Often, use of diuretics to treat ADHF leads to removal of water, but not much sodium. If the salt is not removed, the water is often rapidly reabsorbed. One potential solution for this problem can be direct sodium removal (DSR), such as disclosed in U.S. Publication No. US2018344917, filed May 21, 2018, and published on Dec. 6, 2018, and entitled “Direct Sodium Removal Method, Solution and Apparatus to Reduce Fluid Overload in Heart Failure Patients”. DSR systems propose taking advantage of the well-known phenomenon that “water follows salt.” In the system disclosed in the foregoing US patent publication, a hypotonic (low-sodium) infusate is introduced to the peritoneal cavity. Osmotic pressure draws sodium from the rest of the body into the infusate. The infusate is then removed from the peritoneal cavity by an implanted pump, removing the sodium with it. The body responds to this sodium removal by excreting a corresponding volume of water. In one option, the implanted pump directly transfers the removed infusate to the bladder for excretion through natural processes.

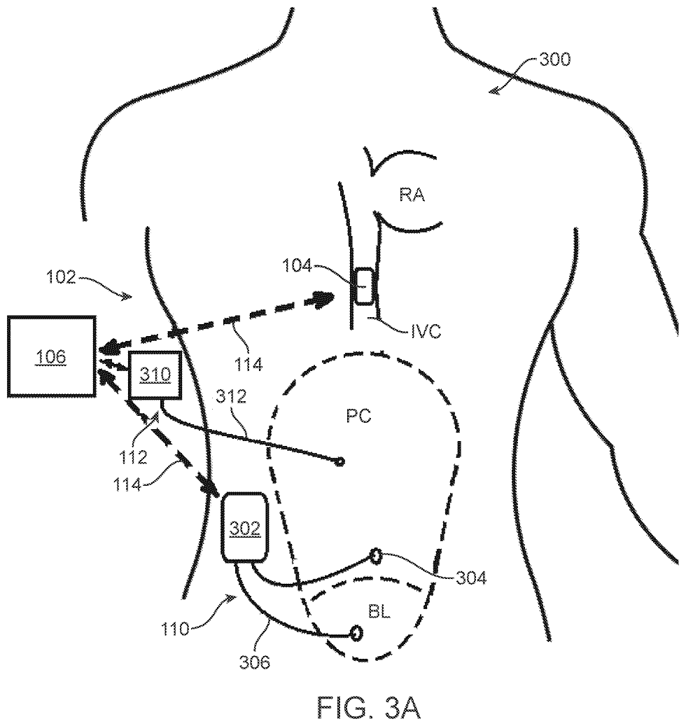

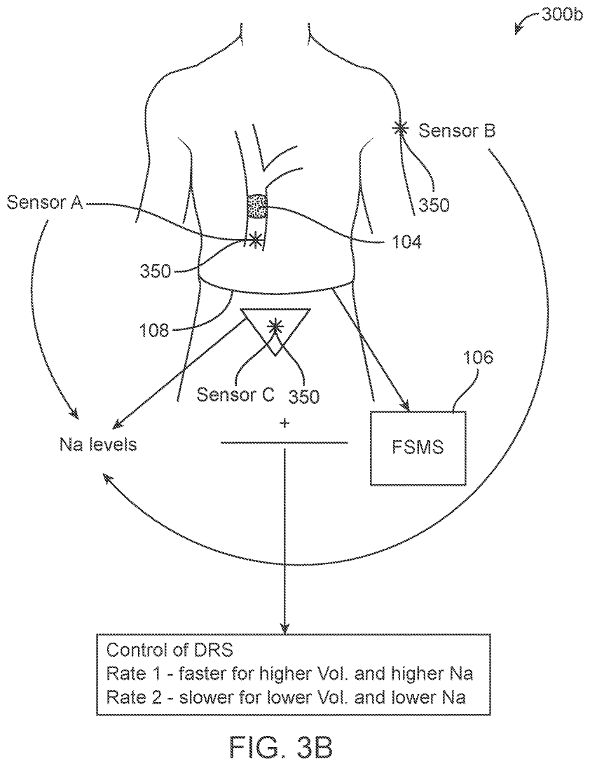

A key challenge in the implementation of the DSR system is determination of blood volume status of the patient as an input into the sodium removal process. FIG. 3A schematically illustrates an embodiment of such a system utilizing the Fluid Status Monitoring Systems described above. In this embodiment, DSR system 300 includes, as an implanted therapy system 110, implantable peritoneal transfer pump 302, with a peritoneal catheter 304 disposed in the peritoneal cavity (PC) and a bladder catheter 306 disposed in the bladder (BL). Fluid Status Monitoring System 102 includes Fluid Status Monitoring sensor 104 (in this example a wireless resonant circuit-based sensor disposed in the patient's IVC, although other disclosed sensor positions and types may be employed). External antenna 108 (see FIG. 1) is omitted in FIG. 3A for clarity.

Fluid Status Monitoring control system 106 communicates through wireless communication channels 114 with implanted pump 302 and with an external therapy system 112, in this case comprising infusion pump 310 and peritoneal infusion catheter 312 (or alternatively syringe delivery of infusate). Integration of Fluid Status Monitoring control system 106 with the DSR system permits optimization of both infusate delivery via pump 310 and excess fluid transfer and removal via implanted pump 302 based on knowledge of the patient's precise blood volume status, thus informing the process parameters such as infusate flow rate or concentration and pump flow rate. In a further alternative embodiment, infusion pump 310 may also be an implanted device rather than an external treatment device.

Another key challenge associated with the DSR system is the determination of the blood sodium levels as an input into the sodium removal system and process. FIG. 3B schematically illustrates an alternative embodiment of DSR System 300b, again including as part of the system a Fluid Status Monitoring System, as described hereinabove. DSR System 300b utilizes a sodium sensor 350 to determine the blood sodium levels as a way to prevent hyponatremia, a low sodium concentration in the blood which is a common occurrence in the use of a DSR system. The sodium sensor 350 and the Fluid Status Monitoring control system 106 controls the amount of fluid removed so as not to lead to low-sodium-associated events including hyponatraemia. If the sodium (Na) sensor 350 detected blood sodium levels below a threshold, set in the system or set individually by the physician, it would then trigger the DSR system to slow the pump speed and thus the rate of sodium removal and vice versa, to manage sodium levels and avoid hyponatraemia.

In one example, system 300b utilizes input from sodium sensor 350, which could be designed and positioned in a number of locations (see FIG. 3B) or could use combinations of these devices and locations: Intravascularly as an implanted sensor 350 measuring blood sodium (sensor position A) Externally as a sensor on the skin providing an alternative way to monitor sodium (sensor position B) Within the bladder to measure the excreted sodium levels (sensor position C)

Hyponatremia is among the most common electrolyte disorders in dialysis patients and there is increasing levels of evidence that suggests that hyponatremia is a risk factor for mortality as well as substantial morbidity, including central nervous system toxicity, hip fracture, immune dysfunction and infection, and cardiovascular complications.

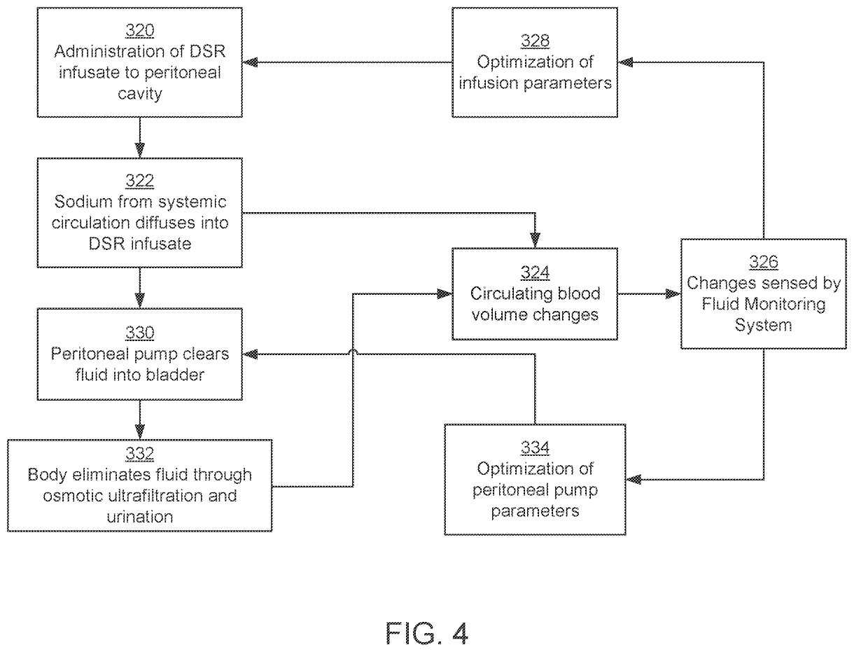

FIG. 4 illustrates a process flow for DSR treatment according to one embodiment disclosed herein. Treatment will typically begin with administration of DSR infusate 320 after which sodium from circulation diffuses into DSR infusate 322. This may lead to increased urination to normalize the salt-water balance, which could then reduce circulating blood volume. Circulating blood volume changes 324 are sensed by the Fluid Status Monitoring System 326 and, in one option, DSR infusion parameters are optimized 328 based on control by the Fluid Status Monitoring System and Sodium Sensing Module. Optimization may include modulation of the protocol for external administration of infusate (via syringe or external infusion pump), or in other alternative embodiments, and may include the control of an implanted pump system to administer infusate with direct input from the Fluid Status Monitoring System. After sodium has diffused into the infusate at 322, the implanted peritoneal pump transfers the fluid from the peritoneal cavity to the bladder 330 and the body eliminates the excess fluid 332. Again, changes in circulating blood volume 324, in this case due to fluid removal, are determined by the Fluid Status Monitoring System 326. Peritoneal pump parameters are thus optimized 334 based on control by the Fluid Status Monitoring System. In this case, examples of optimization include modulation of protocols for external modification of the pump settings by a clinician or direct receipt of the input signal from the Fluid Status Monitoring System and thus direct control of peritoneal pump parameters.

As in the assisted diuresis case above, a key advantage of this novel system is not only the accurate assessment of the intravascular fluid volume but also the extravascular fluid status, detected by the changes in the intravascular volume over time as fluid shifts from extravascular to intravascular and vice versa. For example, if a large net volume of urine is removed but the estimated circulating blood volume does not change, then it is clear that all of this volume was removed from the extravascular compartment.

### IVC or SVC Occlusion for Diuretic Efficiency

In some clinical situations for patients in heart failure, it may be desirable to occlude the IVC below the renal veins in order to improve renal function and diuretic efficiency by reducing the blood pressure in the renal veins. In other situations, occlusion of the SVC may be helpful. A goal is to improve the kidney function by increasing the net perfusion pressure (arterial-venous pressure) to the kidney, thereby increasing renal blood flow and decreasing renal sympathetic activation. Both of these changes can have the effect of increasing urine output. An example of this type of system is disclosed in U.S. Publication No. US2018014829, filed Sep. 27, 2017, and published Jan. 18, 2018, and entitled “Blood Flow Reducer for Cardiovascular Treatment”. However, a challenge with this approach to treatment is to know how much occlusion is required and how to measure the IVC. A number of solutions are possible based on the Fluid Status Monitoring Systems and Devices disclosed herein. For example, placement of a Fluid Status Monitoring Device between the hepatic and renal veins may provide information on IVC geometry, degree of occlusion from flow, renal function and circulating volume refill perspectives.

A number of different embodiments are possible based on disclosed Fluid Status Monitoring Systems. In situations where the patient is a chronic heart failure patient and may already have implanted a Fluid Status Monitoring Device as disclosed, then inputs from Fluid Status Monitoring control system (106 in FIG. 1) may be used as control parameters for the occlusion device. In some cases where it may not be desirable to implant an occlusion device in the IVC long term, such devices would more typically be catheter-based for relatively quick deployment and removal. With the patient catheterized for IVC occlusion, it may be preferable to utilize a catheter-based Fluid Status Monitoring System as disclosed, for example in the present Applicant's incorporated U.S. patent application Ser. No. 15/750,100, filed Aug. 3, 2016 (U.S. Publication No. US20180220992, published Aug. 9, 2019) rather than a wireless implant as elsewhere disclosed. In other cases, an implanted occlusion device may be deemed appropriate and an implantable version of the system described above could be used. Such a fully implanted system may include an occlusion member that would, for example, inflate and deflate automatically in response to signals from the fluid status monitoring device, occluding the flow optimally to facilitate optimal renal function. In such an embodiment, the device may include a reservoir disposed outside the IVC communicating with the inflatable occlusion member inside the IVC via a micro transfer pump controlled by the Fluid Status Monitoring control system 106 or may include a leaflet, tilting disc or other occlusion member, its position relative to the IVC flow modulated by the control system to appropriately occlude IVC flow.

In an alternative embodiment, a combined Fluid Status Monitoring System and IVC Occlusion Device might be implanted within the IVC. A Fluid Status Monitoring System might be implanted in the IVC between the renal veins and hepatic veins, connected directly to an IVC Occlusion Device implanted between the iliac veins and the renal veins. If the IVC Occlusion Device is mechanical and operated using an electrical motor, solenoid, or valve, it could be connected to the Fluid Status Monitoring System with wires. If the IVC Occlusion Device is an inflatable balloon or bladder, the combined device might have a second, longer and relatively low-profile tubular reservoir located near the Fluid Status Monitoring System, perhaps positioned against one wall of the IVC. It would be fluidly connected to the balloon or bladder of the IVC Occlusion Device. A pump located between the reservoir and balloon could transfer fluid from one chamber to the other, to inflate or deflate the IVC Occlusion Device in response to a signal from the Fluid Status Monitoring System. This combined system could be completely implantable and autonomous, or it could rely upon an external controller and/or transcutaneous energy delivery system to wirelessly control and power the system.

In yet another alternative embodiment, the Fluid Status Monitoring System could be combined with a Superior Vena Cava (SVC) Occlusion Device such as that proposed in U.S. Publication No. US20190126014, filed Oct. 23, 2018, and published May 2, 2019, and entitled “Systems and Methods for Selectively Occluding the Superior Vena Cava for Treating Heart Conditions”. Intermittently partially occluding the SVC may cause increased blood flow back to the heart from the IVC. This lowers the IVC pressure, increases renal blood flow, and increases urine output.

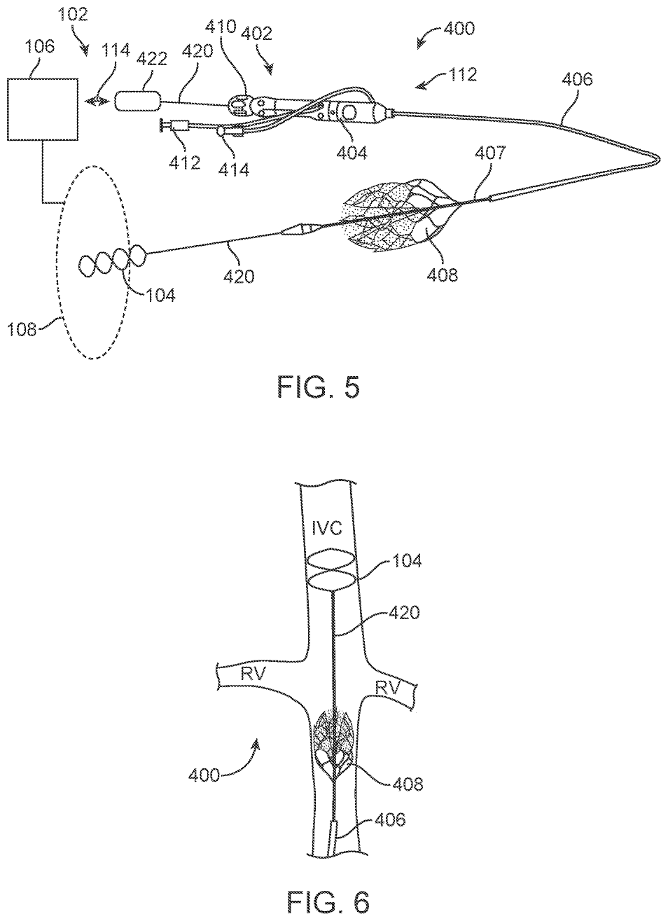

In another alternative embodiment, illustrated schematically in FIGS. 5 and 6, a Fluid Status Monitoring System and device may be incorporated into an IVC occlusion device. As shown therein, system 400 comprises an integrated external treatment device 112 formed as IVC occlusion catheter 402 with distal control handle 404 and catheter body 406. Catheter body 406 defines a lumen that carries delivery member 407 for occlusion device 408. Configuration and operation of catheter body 406 and delivery member 407 may be based on conventional guide catheter/guide wire designs. Occlusion device 408 may be made, for example, from a nitinol frame with a partial polyurethane coating to provide an obstructing member, which may be coated with an anti-thrombotic hydrogel. Occlusion device 408 expands when released from catheter body 406 and collapses by being drawn back into the catheter body. The size of the occlusion device 408, and thus the degree of occlusion, may be adjusted by turning adjustment knob 410 at the distal end of handle 404. Heparin port 412 and peripheral pressure port 414 also may be provided. A cvp port (not shown) through handle 404 and central lumen through delivery member 407 allow for over-the-guidewire placement of occlusion catheter 402, as well as subsequent deployment of catheter-based Fluid Status Monitoring System 102 as described below.

Catheter-Based Fluid Status Monitoring System 102 comprises delivery member 407, which is sized to be received through the cvp port and lumen of catheter 402. Deployable Fluid Status Monitoring Device is disposed at the distal end of delivery member 420 for deployment in the IVC cranially with respect to the renal veins (RV) as shown in FIG. 6. Hub 422 is used for manipulation of delivery member 420 and to provide communication through communication channel 114 with Fluid Status Monitoring control system 106. External antenna 108, which would surround the patient's torso to receive signals from Fluid Status Monitoring Device, also may optionally be included and communicate with Fluid Status Monitoring control system. For example, in embodiments wherein a resonant circuit-based Fluid Status Monitoring Device 104 is used, external antenna 108 would be required. However, where, for example, an ultrasound-based Fluid Status Monitoring Device is employed, then communication with control system 106 may be through a wired communication pathway defined by delivery member 420 and hub 422.

These systems for monitoring circulating blood volume and then intermittently partially occluding the IVC or SVC could significantly enhance diuresis by the kidneys. This would enhance the effectiveness of diuretics, and may enable some patients to reduce or eliminate the use of diuretics entirely. This may have beneficial effects for the patients, as the negative effects of long-term use of high levels of diuretics are well documented.

### Comprehensive Fluid Management System

The management of volume-related diseases could be improved by a better understanding of the status of the balance/location of fluid within the body. Fluid can reside within a number of compartments within the body. In an average adult male, about two-thirds of the fluid volume resides in the intracellular compartment and one-third in the extracellular compartment. Spaces within the extracellular compartment include intravascular and extravascular spaces. As an example, an average 70 kg adult male may have about 14 liters of extracellular fluid, of which intravascular fluid accounts for approximately 3 liters, while extravascular fluid accounts for the remaining 11 liters. Within the intravascular space, the body also has the ability to quickly shift blood between the splanchnic system, holding about 25% of the intravascular volume, and the main circulating volume. Capillary microcirculation and osmotic flow across the vascular walls facilitate shifts between these compartments. Within the extravascular compartment, an excess of extravascular fluid and fluid pressure in tissue is described as edema. In heart failure patients, fluid can collect in the lungs (congestion). In certain patients, extracellular fluid can also collect in the abdominal cavity (ascites). Knowledge of the status of these multiple interconnecting systems is key in the management of patients with fluid-overload-related conditions such as heart failure or those on dialysis or ultrafiltration. More specifically in heart failure, excess intravascular fluid is ultimately transferred to the extravascular space where it can result in buildup of fluid in the lungs. In patients being treated for acute decompensated heart failure (ADHF), removal of excess intravascular fluid may lead to reabsorption of the fluid in the lungs by the circulatory system. However, these patients may still have significant excess extravascular fluid in their tissues which is not removed by the ADHF treatment. After release from the hospital, some of this excess extravascular fluid may migrate into the vascular system and from there into the lungs, leading rapidly to another event of ADHF. It would be very helpful to measure the fluid volumes in as many of the compartments as possible, to have a more comprehensive understanding of the patient's fluid volume status. This would be helpful in the acute setting, in management of ADHF or a specific episode of dialysis, and chronically, to maintain a patient's proper fluid balance and prevent episodes of ADHF.

A wide variety of sensors for measuring extravascular fluid volumes have been proposed, developed, and tested. An example of such an extravascular interstitial fluid sensor is described in U.S. Publication No. US20180271371, filed May 7, 2018, and published Sep. 27, 2018, and entitled “Wireless Interstitial Fluid Pressure Sensor”. Interstitial fluid volumes can also be estimated via measurements within the lymphatic drainage system. Non-invasively, interstitial fluid volume can be estimated by various transdermal microneedle tissue sensors or by pure transdermal measurements of electrical conductivity, dielectric permittivity, analysis of sweat contents, and other means. One such sensor may comprise, for example, a bioimpedance-based fluid sensor such as disclosed in U.S. Publication No. US20190015013, filed Jul. 10, 2018, and published Jan. 17, 2019, entitled “Techniques for Determining Fluid Volumes Using Bioimpedance Information.” To measure extravascular fluid in the lungs, a variety of implantable and wearable thoracic impedance measurement devices have been developed, such as devices disclosed in U.S. Publication No. US20190059777, filed Aug. 28, 2017, and published Feb. 28, 2019, and entitled “Method and System for Determining Body Impedance,” and U.S. Publication No. US20130281800, filed Jun. 20, 2013, and published Oct. 24, 2013, and entitled “Method, System and Apparatus for Using Electromagnetic Radiation for Monitoring a Tissue of a User.”

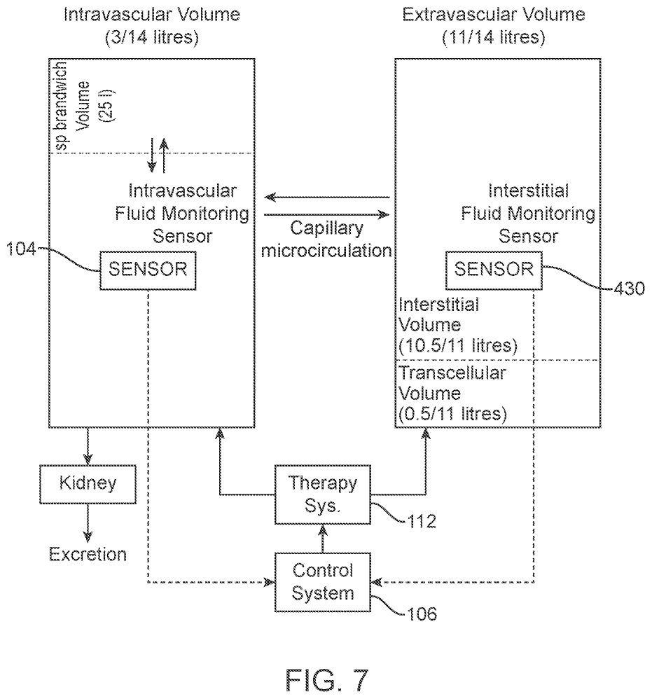

FIG. 7 shows a schematic depiction of a system to monitor the distribution of fluid across the extracellular compartment and between the intravascular and extravascular spaces. In one embodiment, two or more sensors monitor intravascular and extravascular fluid status output signals to inform the overall fluid balance. As schematically depicted in FIG. 7, intravascular sensor 104, examples of which are discussed above, communicates with control system 106. Extravascular interstitial fluid sensor 430 also communicates with control system 106. Control system 106 may optionally control therapy delivered via therapy system 112 based on fluid status information received from each of the sensors. Alternatively, or additionally, an implanted therapy delivery system 110, as shown in FIG. 1, may be used and communicate with control system 106.

Intravascular fluid status monitoring sensor 104 provides information on the status of the fluid volume within the vessel in which it is placed, for example the IVC, and based thereon an accurate estimation of the intravascular circulating volume. Interstitial fluid monitoring sensor 430, used to measure interstitial or extravascular volume, may comprise any one or more of the sensors described above. These two sensor inputs (intravascular and extravascular) may be combined and analyzed in control system 106 in accordance with predetermined algorithms. In one embodiment, a single cloud-based analysis system may be used as control system 106 to manage comprehensive fluid status. The knowledge of the system balance as determined by control system 106 could then be used to better inform therapeutic decision making to optimize treatment on a personalized, individual basis and could be used to optimize therapy (diuresis/dialysis, etc.).

One application of this Comprehensive Fluid Management System is in chronic monitoring of heart failure patients. For example, it might be determined that although intravascular fluid volume is stable and at an acceptable level, extravascular fluid volume is gradually increasing, and therefore it makes sense to adjust the patient's care (such as increasing diuretic dose). This might decrease intravascular fluid volume, which causes some of the excess extravascular fluid to migrate into the intravascular compartment. Alternatively, extravascular volume may be decreasing, leading to dehydration, despite a normal intravascular fluid volume. Undetected dehydration is a major problem in the elderly, leading to hundreds of thousands of hospital visits every year. In this instance, the patient could reduce the dose of diuretics they are taking, if any, and/or drink additional rehydration fluids to restore their extravascular fluid level.

A second application of this Comprehensive Fluid Management System is in the management of dialysis patients. Since these patients are generating little or no urine with their kidneys, they are in a state of fluid overload every few days, and that fluid needs to be removed via dialysis. However, the dialysis process typically only lasts a few hours. During this period, the excess intravascular fluid is removed, and the intravascular sensor 104 can be used to manage the removal of as much excess intravascular fluid as possible without dangerously depleting intravascular fluid volume. At the same time, the extravascular interstitial fluid sensor 430 can monitor the extravascular volume, determining how much excess extravascular volume is present and how quickly it is migrating into the intravascular compartment. This extravascular monitoring may indicate when it is necessary to prolong the dialysis process after the toxins and excess intravascular fluids have been removed, in order to allow excess extravascular fluids to migrate into the extravascular space and be removed as well.

A third application of this Comprehensive Fluid Management System is in the management of acute decompensated heart failure (ADHF) patients. As mentioned above, patients undergoing treatment for ADHF are often discharged from the hospital or end treatment before sufficient excess fluids or salt have been removed. This often occurs in part because treatment with aggressive doses of diuretics is ineffective in generating additional urine production. Often the acute therapy removes the excess intravascular volume and the extravascular volume in the lungs, but not the rest of the excess extravascular volume in other tissues of the body. Since 80% of the fluid in the body is extravascular, if even a portion of the excess extravascular fluid migrates into the intravascular space, then the patient can very quickly return to a state of ADHF. Therefore, monitoring extravascular fluid volume as well as intravascular volume, and making sure that extravascular fluid volume is appropriately reduced, could dramatically improve the management and effectiveness of the fluid removal process.

Heart failure decompensation is often associated with the transition of fluid from intravascular, as monitored by sensor 104, to extravascular, as monitored by sensor 430 and ultimately ends up as fluid in the lungs leading to shortness of breath and acute presentation in the hospital. This combined sensor system would facilitate management of this complex biological system to guide patient care and avoid acute decompensations.

Another heart failure scenario in which this system can provide vital information is when this transfer of fluid from intravascular to extravascular ends up leading to ascites or the abnormal accumulation of fluid in the abdomen. This can lead to poor digestive performance and the subsequent poor uptake of orally administered diuretic medication, resulting in the spiraling worsening of patient symptoms due to systemic fluid retention, ultimately leading to decompensation and admission to hospital for intravenous diuretic administration. This system could prevent the onset of ascites through knowledge of the balance of intravascular and extravascular fluid statuses.

### Lymphatic Drainage

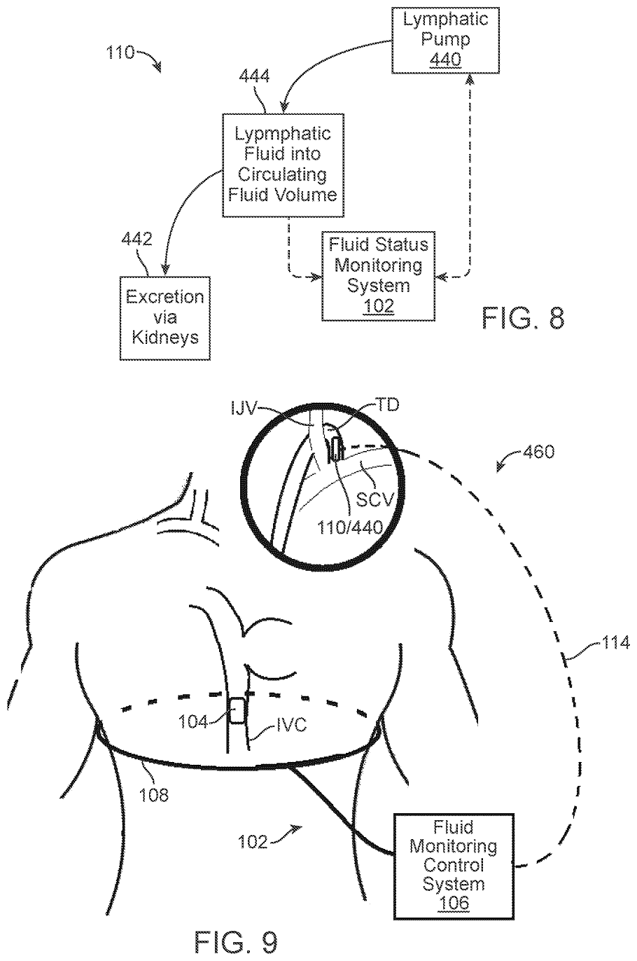

In other embodiments, fluid balance may be modulated by augmenting natural drainage through the lymphatic system. The block diagram of FIG. 8 illustrates an example of such an approach. In such embodiments an implanted therapy system 110 may comprise a wirelessly controlled lymphatic pump 440, which assists the natural lymphatic system in delivering excess fluid into the circulating blood volume at 444 for natural excretion via the kidneys at 442. Alternatively, rather than a wireless lymphatic assist pump, a catheter-based system, such as disclosed, for example in U.S. Pat. No. 9,901,722, granted Feb. 27, 2018, and entitled “System and Method for Treatment of Pulmonary Edema,” may be employed. In the case of use of a catheter-based lymphatic assist device, it may be preferable to utilize catheter-based embodiments of fluid status monitoring system 102 as described above.

FIG. 9 schematically depicts a wireless system 460 for lymphatic assist as means for modulating patient fluid balance. In one embodiment, wireless implanted lymphatic assist pump 440 is positioned in the thoracic duct (TD) adjacent the branch of the internal jugular vein (IJV) and subclavian vein (SCV) to drain lymphatic fluid into the circulating volume and ultimately out of the body via the kidneys. Other suitable locations for pump 440 may be devised by persons skilled in the art based on the teachings of the present disclosure. In this example, fluid status monitoring system 102 utilizes wireless monitoring device 104 implanted in the IVC and communicates with fluid monitoring control system 106 via external antenna 108 around the patient's chest. Control system 106 communicates with implanted pump 440 via wireless communications link 114, which allows for optimization of pump operating parameters based on patient fluid status as determined by direct measurement of the IVC volume. In one operating mode, by maintaining a slightly hypotensive or hypovolemic state, without allowing severe hypotension or hypovolemia, lymphatic flow may be optimized.

### Diaphragm Stimulation

Phrenic nerve stimulation causing diaphragmatic movement has been investigated in the treatment of sleep apnea and heart failure patients. A pulse generator is implanted in the patients and a lead positioned in the left pericardiophrenic or right brachiocephalic vein in order to stimulate the phrenic nerve. This stimulation causes diaphragm contraction similar to that seen in normal breathing. The system is designed to stimulate the diaphragm at night when the patient is sleeping. The interaction between breathing, cardiac output, intravascular pressures and IVC volumes is accepted to be clinically relevant, but not well understood.

These stimulation systems do not have any ability to evaluate the intravascular fluid status of the patient as an input into the therapy. The combination of these systems with the aforementioned Fluid Management System 102 (FIG. 1) including Fluid Status Monitoring device 104 would enable the combined system to be adjusted based on the intravascular fluid status of the patient and therefore optimize their therapy.

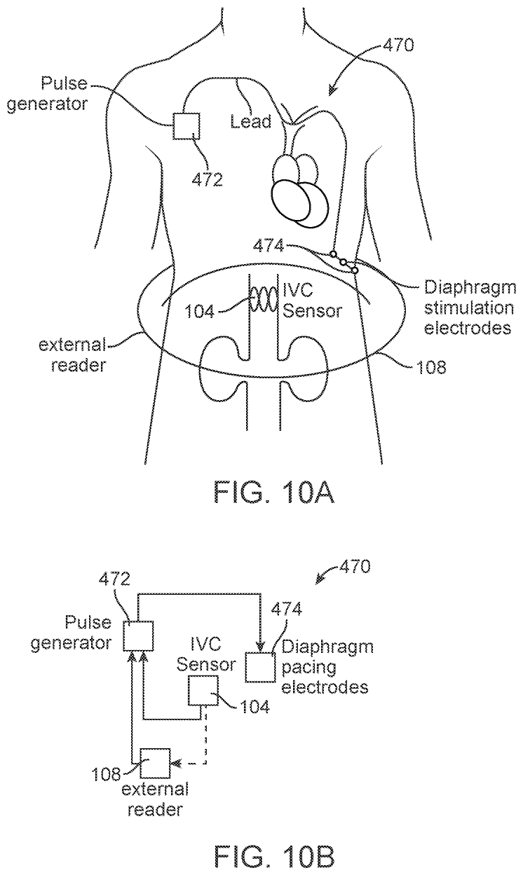

FIGS. 10A and 10B schematically depict a wireless system 470 for diaphragm stimulation with fluid monitoring input, as means for treating heart failure or sleep apnea patients. In one embodiment, diaphragm stimulation pulse generator 472 is positioned in the subclavian space and a lead 474 positioned via vascular access to the left pericardiophrenic or right brachiocephalic vein. The distal end of the lead 474 is positioned such that the electrodes are capable of pacing the phrenic nerve and therefore exciting the diaphragm. Fluid Status Monitoring device 104, as described previously, is capable of detecting an IVC area and therefore fluid volume. This information is relayed to the pulse generator either directly via Bluetooth or other communication protocol or via an externally worn belt antenna 108.

### Vascular Flow Assist

Another interventional device used in treating patients at various stages of heart failure is an implanted or catheter-based pump to assist vascular flow. Treatment with such assist pumps may be more closely matched to patient clinical need by modulating pump operation based on real-time patient fluid status information as determined by Fluid Management System 102 when used as a control input to the assist pump. One type of assist system that would be beneficially augmented with control based on signals from a Monitoring Device 104 is an intracardiac pump used to support the natural pumping function of the heart. One example of an intracardiac pump is described in US Published Patent Application No. US2010/026801 A1, filed Jan. 6, 2006 (now U.S. Pat. No. 9,872,948), entitled “Intracardiac Pumping Device.”

Therapeutic vascular assist pumps are also proposed to be implanted in the IVC or renal veins to increase flow rate in the IVC and reduce the renal vein pressure, thus mechanically unloading the kidneys and assisting kidney function. Such therapies may also lead to the excretion of more fluid from the body and lead to changes in patient fluid state. However, current systems do not include an ability to evaluate the intravascular fluid status of the patient as an input into the therapy. Current methodologies for fluid state monitoring, such as measuring urine output and/or blood pressure monitoring, may not provide sufficiently real-time information on patient fluid state to adequately facilitate system modulation. To avoid fluid imbalance when utilizing such therapies, it would be preferable to modulate system operation with input based on accurate patient fluid state information. The combination of these systems with the aforementioned Fluid Management System 102 (FIG. 1) including Fluid Status Monitoring device 104 would enable the combined system to be adjusted based on the intravascular fluid status of the patient and therefore optimize their therapy.

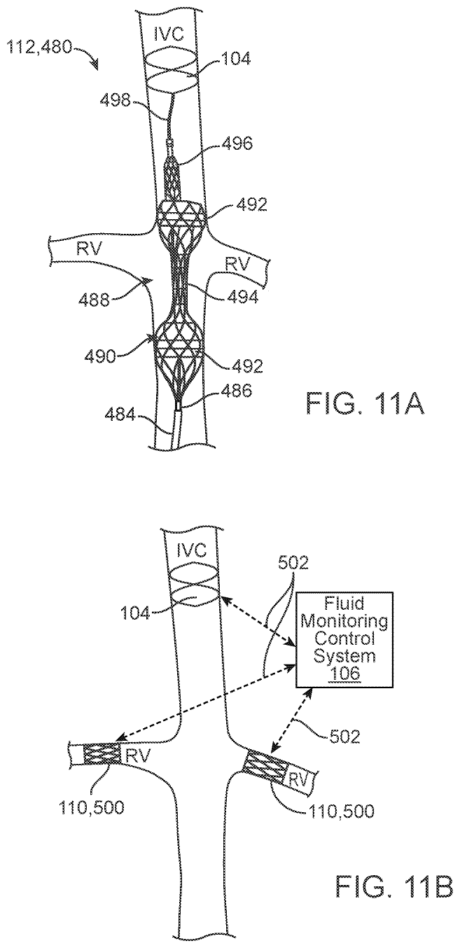

Examples of such combined systems are shown in FIGS. 11A and 11B. FIG. 11A shows an external therapy device 112 configured as catheter-based IVC assist pump system 480. As shown therein, external therapy device 112/480 includes guide catheter 484 for guiding flexible elongate delivery member 486 to a position adjacent the therapy site in the IVC. Delivery member 486 has disposed at its distal end, expandible and collapsible pump system 488. Pump system 488 comprises a body member 490 surrounded by an expandable cage. In some embodiments the body member may also include flexible covering to provide IVC occlusion. One or more collapsible impeller pumps 492 are disposed within body member 490. Central portion 494 may include a portion of the expandable cage as shown or may comprise only a portion of delivery member 486 and may include sensors such as a pressure sensor. Distal end 496 has an atraumatic configuration and may include additional flow or pressure sensors. Distal end 496 also provides a distal port to central lumen (not shown) for passage of sensor delivery member 498 which has an embodiment of Fluid Status Monitoring Device 104 distally disposed thereon. Pumps 492 and Monitoring Device 104 may communicate with a control system such as Fluid Monitoring Control System 106 (e.g., FIG. 1) through wired connections in delivery members 486 and 498.

FIG. 11B illustrates an example of an alternative renal assist system using wireless implanted therapy devices/renal assist pumps 500. Renal assist pumps 500 communicate via wireless communication links 502 with Fluid Monitoring Control System 106. Another wireless communication link 502 also provides communication between the control system and wireless Fluid Status Monitoring Device 104 as previously described. Examples and more details of renal assist pumps for positioning in the IVC or renal veins are shown in, for example, U.S. Patent Publication No. US20200254161A1, filed Apr. 27, 2020, and published Aug. 13, 202, entitled “Blood Pump”, and U.S. Patent Publication No. US20200,069857A1, filed Nov. 8, 2019, and published Mar. 5, 2020, entitled “Venal-Caval Blood Pump”.

In systems 112/480 and 110/500, wired or wireless Fluid Status Monitoring Device 104 is used to determine fluid status by measuring dimensional parameters of the IVC (as previously disclosed). Thus, using fluid monitoring system 106 as described hereinabove, communicating with the respective pump systems, the performance (e.g., speed and duration of operation) of the pumps is controlled using the IVC data from monitoring device 104 as an input. Renal assist systems as disclosed herein would thus operate based on the IVC dimensional monitor 104 providing an output signal which is communicated (via Bluetooth®, wi-fi or other means) to the renal decongestion pump. The pump receives this signal via a powered communications module, processes the signal and determines what action/therapy to take based on an algorithm. The pump-operating parameters would be controlled to manage the pump performance in response to the IVC measurement signals. These parameters may include the speed and operation duration of the pump.

### Diuretic Patch Pump

Advances in micro-pump technology have made wearable patch pumps a practical alternative for automated ambulatory drug dosing. Embodiments disclosed herein utilize such patch pumps for drug delivery therapies for diuretic, vasodilator or other heart failure medication or combination of medications. For example, Fluid Status Monitoring Device 104 communicating through control system 106 may be used to determine fluid status by measuring dimensional parameters of the IVC (as previously disclosed). This information is combined as an input to the patch pump to deliver a therapeutic agent. The response of the body to this is monitored using the IVC sensor and the delivery of the therapeutic agent modified as required based on a pre-programmed algorithm.

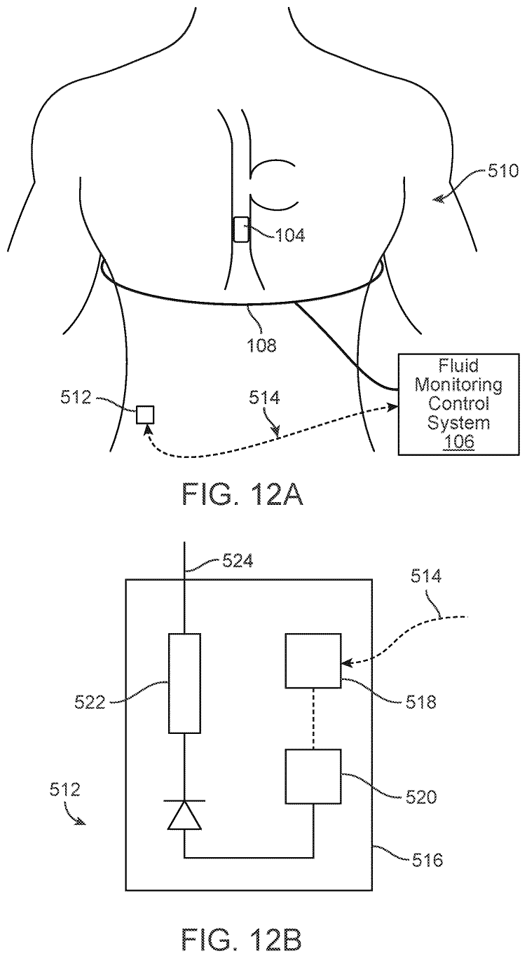

FIGS. 12A and 12B illustrate an embodiment of such a patch pump system. As shown therein, system 510 employs patch pump 512 communicating with control system 106 via wired or wireless communications link 514. As previously described, control system 106 also communicates with and energizes monitoring device 104 via belt antenna 108. Communications link 514 may be via Bluetooth®, wi-fi or other means. Patch pump 512, as shown in FIG. 12B, includes housing 516 with a skin adherable surface. Contained within or supported by housing 516 are components such as processor/power module 518, drive motor 520, pump module 522 and needle 524. Pump module 522 contains the therapeutic agent and may comprise a variety of micro-pump technologies such as syringe pumps or micro-rotary piston pumps. Further details of suitable pumps are described for example in US Patent Publication No. US20190358393A1, filed Nov. 7, 2017, and published Nov. 28, 2019, entitled “Drug Delivery Device”.

In operation, pump 512 receives a signal via communications link 514 and processes the signal representing patient fluid state and determines what action to take based on a drug delivery algorithm. In alternative embodiments, the drug delivery algorithm is contained within and executed by control system 106 and the control signal to patch pump 512 then comprises a direct pump control signal. As will be appreciated by persons skilled in the art, while these embodiments are exemplified by a patch pump, the systems described above could also comprise a subcutaneous implanted pump with the same control system.

Each of the patents and published patent applications cited hereinabove are incorporated herein by reference in its entirety.

The foregoing has been a detailed description of illustrative embodiments of the disclosure. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure or of the inventions as set forth in following claims.

What is claimed is:

1. An intracardiac interventional assistance system for patient fluid management comprising:
a cardiac pump configured to output blood, wherein the cardiac pump is configured to be implanted in a heart or to be a catheter-based pump to assist with cardiac output;
a vascular dimension monitoring sensor device configured to be positioned within a vena cava vascular lumen, wherein the vascular dimension monitoring sensor device is configured to change resonant frequency of a vascular dimension monitoring sensor at a monitored location of the vena cava vascular lumen, the vascular dimension monitoring sensor device configured to be secured at the monitored location to change the resonant frequency without affecting the natural dilation and constriction of the vena cava vascular lumen;
an external processing system in electronic communication with the vascular dimension monitoring sensor device, the external processing system comprising an external receiver configured to receive from the vascular dimension monitoring sensor device data comprising the changes detected in the resonant frequency, the external processing system configured to determine changes in area of the vena cava vascular lumen based on the changes detected in the resonant frequency, wherein the changes determined in the area of the vena cava vascular lumen are based on changes in a patient fluid state; and
an electronic control system in electronic communication with the cardiac pump and the external processing system, the electronic control system configured to control the cardiac pump to modulate a flow of output blood from the cardiac pump based on the changes determined in the area the vena cava vascular lumen by the external processing system, wherein the control of the cardiac pump by the electronic control system is configured to substantially match in real-time a clinical need of the patient generated in part by the changes determined in the area of the vena cava vascular lumen, wherein the clinical need of the patient is determined based at least in part on a circulating blood volume.

2. The system of claim 1, wherein the cardiac pump comprises at least one impeller.

3. The system of claim 1, wherein the cardiac pump is collapsable.

4. The system of claim 1, wherein the cardiac pump comprises an expandable cage.

5. The system of claim 1, wherein the vascular dimension monitoring sensor device is further configured to communicate with the cardiac pump directly.

6. The system of claim 1, wherein the vascular dimension monitoring sensor is further configured to detect the changes in lumen diameter in substantially real time.

7. The system of claim 1, wherein the cardiac pump further comprises at least one pressure sensor.

8. An intracardiac interventional assistance system for patient fluid management comprising:
a cardiac pump configured to output blood, wherein the cardiac pump is configured to be implanted in a heart or to be a catheter-based pump to assist with cardiac output;
a vascular monitoring sensor device configured to be positioned within a vessel, wherein the vascular monitoring sensor device is configured to detect hemodynamic changes with a vascular monitoring sensor at a monitored location, the vascular monitoring sensor device configured to be secured at the monitored location without affecting or obstructing the natural dilation and constriction of the vessel;
an external processing system in electronic communication with the vascular monitoring sensor device, the external processing system comprising an external receiver configured to receive from the vascular monitoring device data comprising the hemodynamic changes, the external processing system configured to determine changes in a patient volume status based on the changes detected by the vascular monitoring sensor device; and
an electronic control system in electronic communication with the cardiac pump and the external processing system, the electronic control system configured to control the cardiac pump to modulate a flow of output blood from the cardiac pump based on the hemodynamic changes and the patient volume status, wherein the control of the cardiac pump by the electronic control system is configured to substantially match in real-time a clinical need of the patient generated in part by the changes determined in the patient volume status, wherein the clinical need of the patient is determined based at least in part on hemodynamic changes.

9. The system of claim 8, wherein the cardiac pump comprises at least one impeller.

10. The system of claim 8, wherein the cardiac pump is collapsable.

11. The system of claim 8, wherein the cardiac pump comprises an expandable cage.

12. The system of claim 8, wherein the vascular monitoring sensor device is further configured to communicate with the cardiac pump directly.

13. The system of claim 8, wherein the vascular monitoring sensor is further configured to detect the changes in lumen diameter in at least near-real time.

14. The system of claim 8, wherein the cardiac pump further comprises at least one pressure sensor.

15. A computer-implemented method for assisting patient fluid management via an intracardiac interventional assistance system, the computer-implemented method comprising:
electronically receiving, by a receiver of an external processing system from a vascular monitoring sensor device, data comprising hemodynamic changes, the hemodynamic changes detected at a monitored location within a vessel by a vascular monitoring sensor of the vascular monitoring sensor device, the vascular monitoring sensor device configured to be secured at the monitored location within the vessel without affecting or obstructing natural dilation and constriction of the vessel;
determining, by the external processing system, changes in a patient volume status based on the changes detected by the vascular monitoring sensor device;
generating, by the external processing system, a desired flow of output blood from a cardiac pump based on the hemodynamic changes and the patient volume status, and
electronically transmitting, by a transmitter of the external processing system, the desired flow of output blood from the cardiac pump to an electronic control system in electronic communication with the cardiac pump, the electronic control system configured to control the cardiac pump to modulate a flow of output blood from the cardiac pump to the desired flow of output blood, wherein the control of the cardiac pump by the electronic control system is configured to substantially match in real-time a clinical need of the patient generated in part by the changes determined in the patient volume status, wherein the clinical need of the patient is determined based at least in part on hemodynamic changes,
wherein the external processing system comprises a computer processor and an electronic storage medium.

16. The computer-implemented method of claim 15, wherein the hemodynamic changes are detected at the monitored location in substantially real-time following the natural dilation and constriction of the vessel.

17. The computer-implemented method of claim 15, wherein the cardiac pump is implanted in a heart or is a catheter-based pump.

18. The computer-implemented method of claim 15, wherein the electronic control system communicates wirelessly with the cardiac pump.

19. The computer-implemented method of claim 15, wherein the cardiac pump further comprises at least one pressure sensor.

20. The computer-implemented method of claim 15, wherein the cardiac pump occludes the vessel.
